(19) United States
(12) Patent Application Publication
(10) Pub. No.: US 2017/0303388 A1
(43) Pub. Date: Oct. 19, 2017

(54) FLAT CABLE STRAIN RELIEF WITH CONTROLLED MECHANICAL RESISTANCE

(71) Applicant: KONINKLIJKE PHILIPS N.V., EINDHOVEN (NL)

(72) Inventor: Brian Bernard MATHEWSON, ROCKY RIVER, OH (US)

(21) Appl. No.: 15/510,760

(22) PCT Filed: Sep. 7, 2015
(86) PCT No.: PCT/IB2015/056819

§ 371 Date: Mar. 13, 2017

Related U.S. Application Data
(60) Provisional application No. 62/053,930, filed on Sep. 23, 2014.

(51) Int. Cl. H05K 1/02; A61B 6/00; H01B 7/08; A61B 6/03

(52) U.S. Cl. CPC H05K 1/028; H01B 7/08; A61B 6/56; A61B 6/589; A61B 6/037; H05K 2201/09027; H05K 2201/09063; H05K 2201/10189

(57) ABSTRACT

A flexible electronic pathway, such as a flat flexible cable or flexible circuit, includes a cut-out within the pathway to provide strain relief by balancing the stress over the termination area. The flexible electronic pathway allows relative movement between a first end and a second end in all three translation, all three rotation directions, and combinations thereof. The strain relief can provide for a controlled mechanical resistance to reduce the risk of damage and failure.

Flexible electronic pathways allow for electricity to flow through pathways that require flexibility, including, for example, where there is relative movement between ends of the electronic pathway or the ends of a conductor facilitating the electronic pathway. Flat flexible cables, flexible circuits, polymer thick film (PTF) circuits, and flexible conductor sheets are common examples of flexible electronic pathways. Flat Flexible Cables (FFCs) are typically cables composed of one or more layers of plastic and conductive circuits. FFCs are commonly used to provide mass terminations between printed circuit board (PCB) assemblies, LCD display panels, sensors, etc. FFCs may be chosen due to their space-saving attributes and low cost. Flex circuits, like FFCs, can feature conductive traces but often incorporate attached components. (FFC will be used herein to refer to both flex cables, flex circuits, PTF circuits, and flex sheets.) FFCs are typically terminated at PCBs by connectors or directly to the PCB, for example, using soldering or anisotropic conductive film (ACF) tape. While most of these connections are internal to end products, stress can be placed on the terminations due to various conditions, including, for example, assembly, hardware upgrades, service, handling, vibration, thermal expansion, etc.

Damage or disconnection at an FFC termination can occur when forces, such as, for example, pulling, pushing, lifting, twisting, combinations thereof, etc., are applied to the FFC since there is often nothing but the termination itself to secure the FFC. FFC to PCB connections are especially susceptible when terminations are made directly to the PCB with ACF tape adhesive. Once such a termination is damaged, options for repair are often limited because of the specialty equipment required to bond them.

Various means for limiting stress and strain on FFCs are found in commercial products. In some cases extra cable length is extended well beyond the required length so that assembly and disassembly of the product can be performed with less risk of applying stress to one side of the cable or the other. When space is limited, excess slack in cables is sometimes taken up by forming folds in the cable, although this is not practical for multilayer FFCs due to the risk of damage. A sharp back-and-forth “Z” crease in a flex cable is sometimes used in products and provides some controlled resistance like a spring, but this effect is functional primarily in a single direction. Sometimes one-sided bends or jogs are incorporated into designs, although these can lead to unbalanced forces at the termination.

In other cases beads of adhesive such as hot glue are applied over the FFCs to secure them. Mechanical features are sometimes incorporated into a product design to secure the FFCs. Both have drawbacks in adding expense and complicating serviceability and are not always feasible given space and manufacturability constraints.

The present application relates generally to a method and system for stress and/or strain relief incorporated into the design of a flexible electronic pathway, including, for example, a FFC, to reduce the rate at which force is applied to the flexible electronic pathway terminations and to balance the forces applied to the ends at the termination sites, effectively providing a controlled mechanical resistance to reduce the risk of damage and/or failure. The methods and systems can provide strain relief in all three translation, all three rotation directions, and combinations thereof. The strain relief features are incorporated into the design of the pathway itself via a cut-out and do not require external means of fixation. The design approach minimizes the area required for the strain relief features and balances stress over the termination area.

The methods and systems are useful in all applications that utilize electronic pathways, such as, for example, FFCs. In one embodiment, methods and systems find particular application with medical diagnostic imaging systems. It is to be appreciated, however, that the invention is also applicable to a wide range of electronics, including various imaging equipment and techniques, for example ultrasonic and magnetic resonance imaging devices, x-ray, computed tomography (CT), positron emission tomography (PET), single photon emission computed tomography (SPECT), etc.

In one embodiment, a flexible electronic pathway includes a flat conductor including an electrical conductor and an insulative substrate, and a cut-out within the flat conductor to provide strain relief, wherein the flexible electronic pathway allows relative movement between a first end and a second end of the flat conductor.

Numerous advantages and benefits will become apparent to those of ordinary skill in the art upon reading the following detailed description of several embodiments. The invention may take form in various components and arrangements of components, and in various process operations and arrangements of process operations. The drawings are only for the purpose of illustrating many embodiments and are not to be construed as limiting the invention.

The descriptions of the invention do not limit the words used in the claims in any way or the scope of the claims or invention. The words used in the claims have all of their full ordinary meanings.

In the accompanying drawings, which are incorporated in and constitute a part of the specification, embodiments of the invention are illustrated, which, together with a general description of the invention given above, and the detailed description given below, serve to exemplify embodiments of this invention, including the methods.

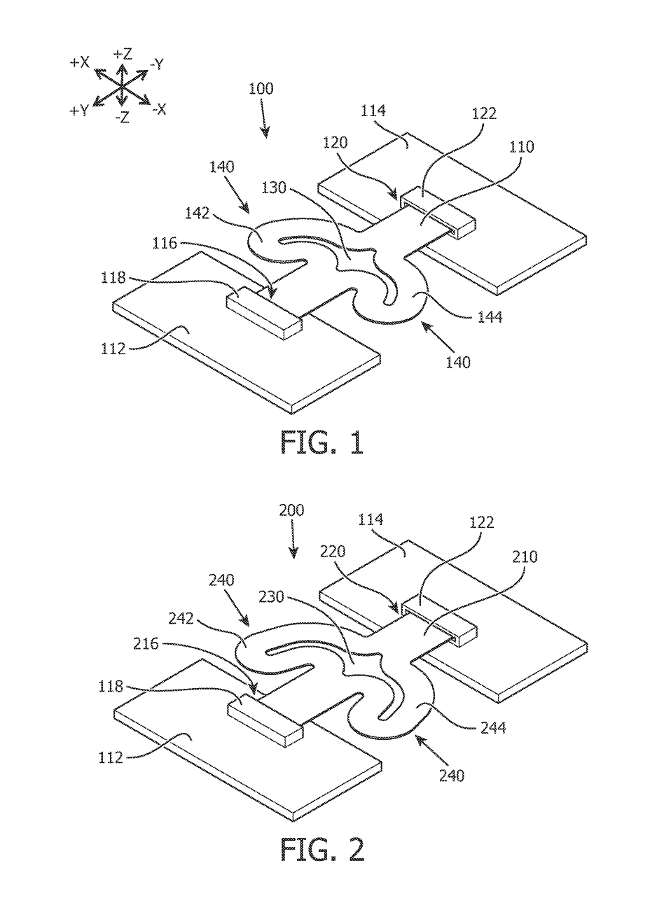

FIG. 1 illustrates an exemplary flexible electronic pathway connecting exemplary devices;

FIG. 2 illustrates another exemplary flexible electronic pathway connecting exemplary devices;

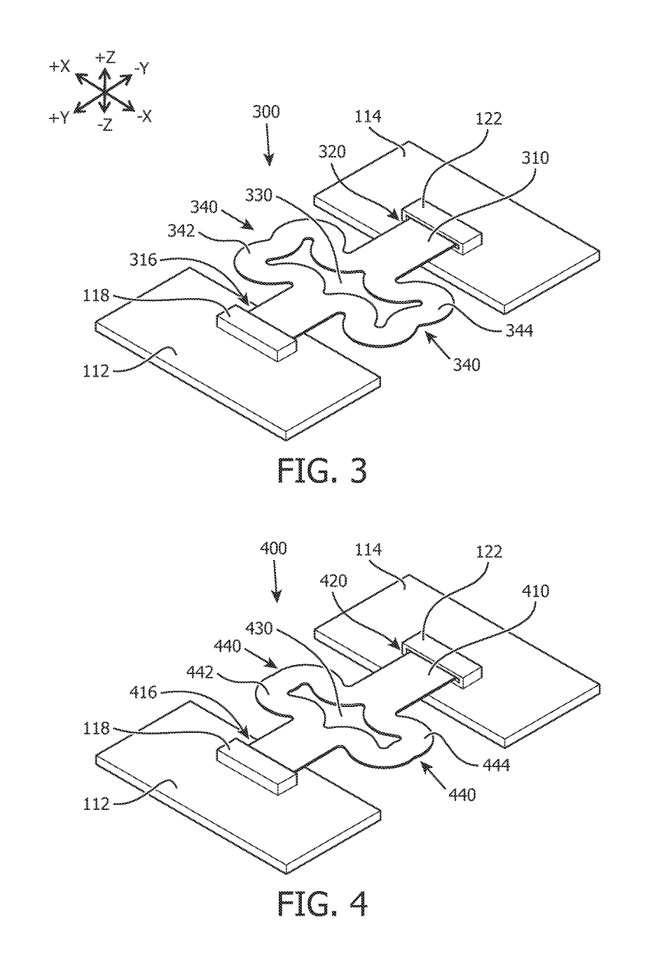

FIG. 3 illustrates another exemplary flexible electronic pathway connecting exemplary devices;

FIG. 4 illustrates another exemplary flexible electronic pathway connecting exemplary devices;

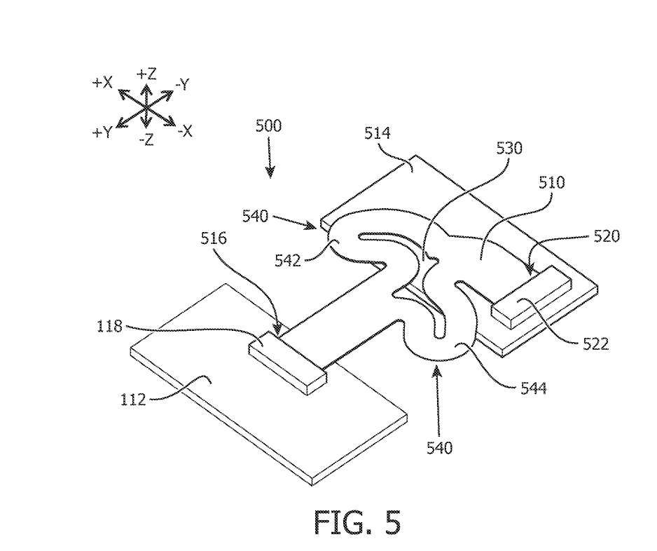

FIG. 5 illustrates another exemplary flexible electronic pathway connecting exemplary devices;

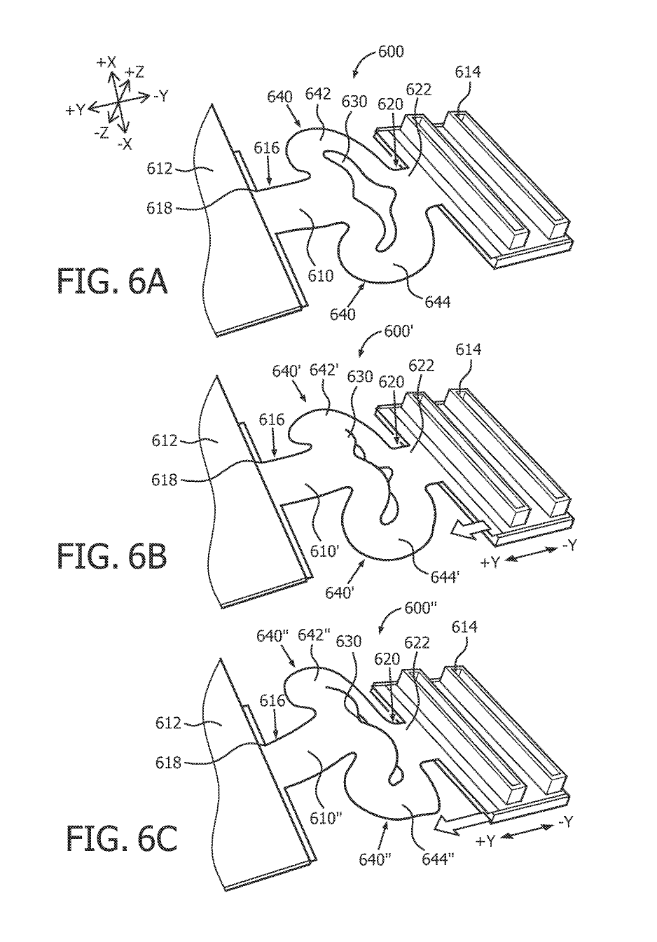

FIG. 6A shows an exemplary flexible electronic pathway connecting exemplary devices in a relaxed state;

FIG. 6B shows the exemplary flexible electronic pathway of FIG. 6A in a tense state;

FIG. 6C shows the exemplary flexible electronic pathway of FIG. 6A in another tense state;

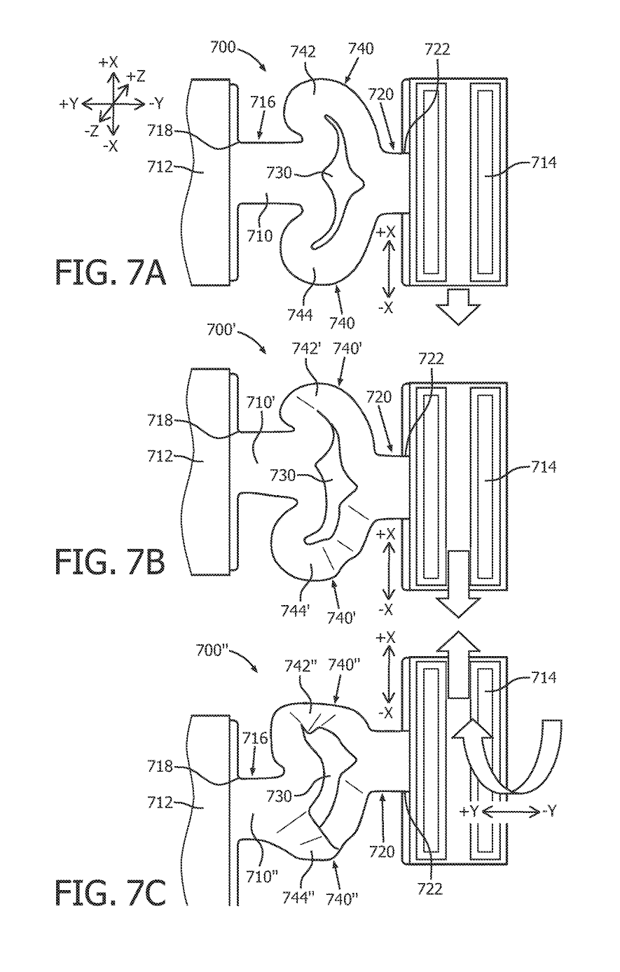

FIG. 7A shows an exemplary flexible electronic pathway in a tense state;

FIG. 7B shows the exemplary flexible electronic pathway of FIG. 7A in another tense state;

FIG. 7C shows the exemplary flexible electronic pathway of FIG. 7A in another tense state;

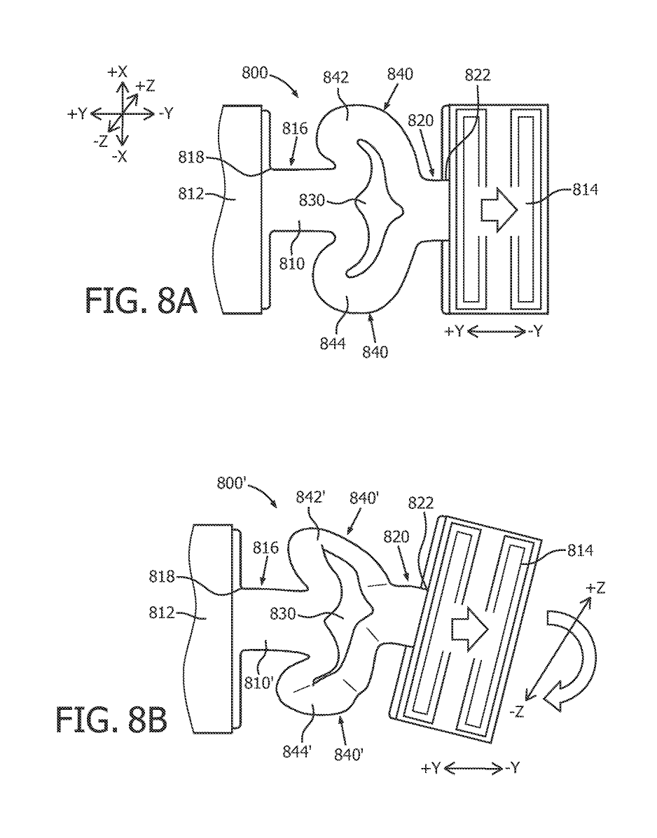

FIG. 8A shows an exemplary flexible electronic pathway in a tense state;

FIG. 8B shows the exemplary flexible electronic pathway of FIG. 8A in another tense state;

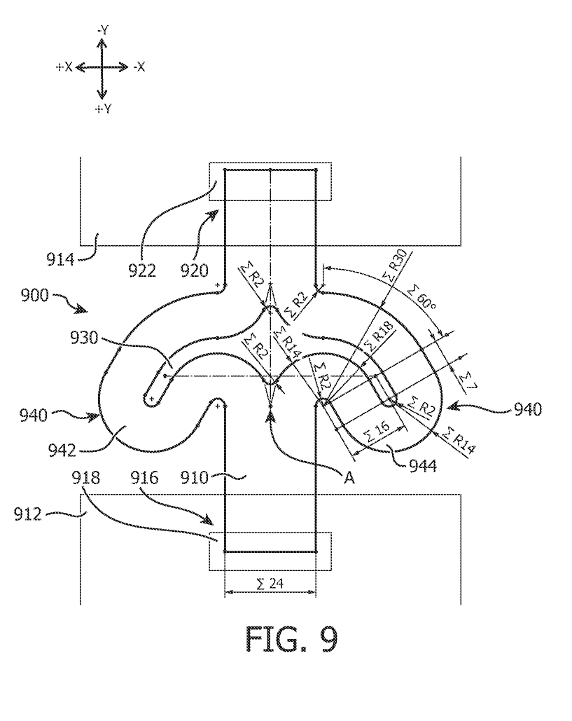

FIG. 9 illustrates an exemplary flexible electronic pathway connecting exemplary devices with specific exemplary design dimensions;

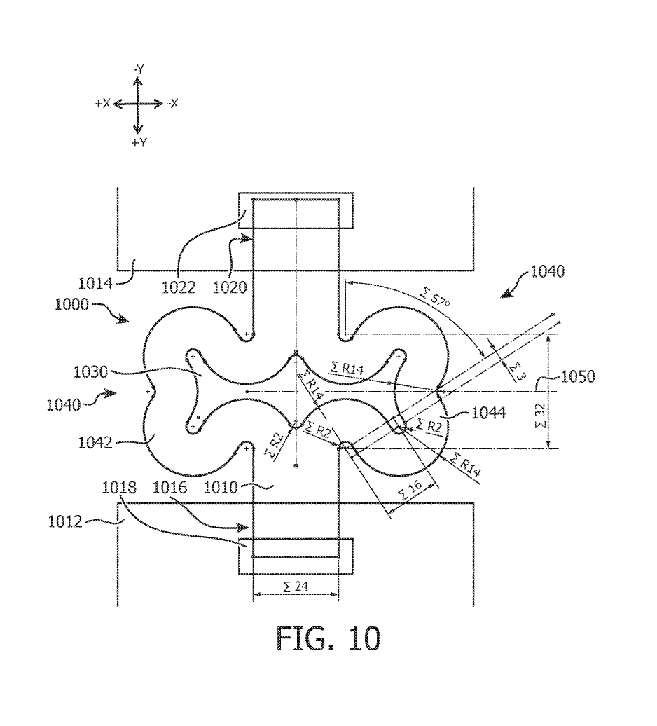

FIG. 10 illustrates another exemplary flexible electronic pathway connecting exemplary devices with specific exemplary design dimensions;

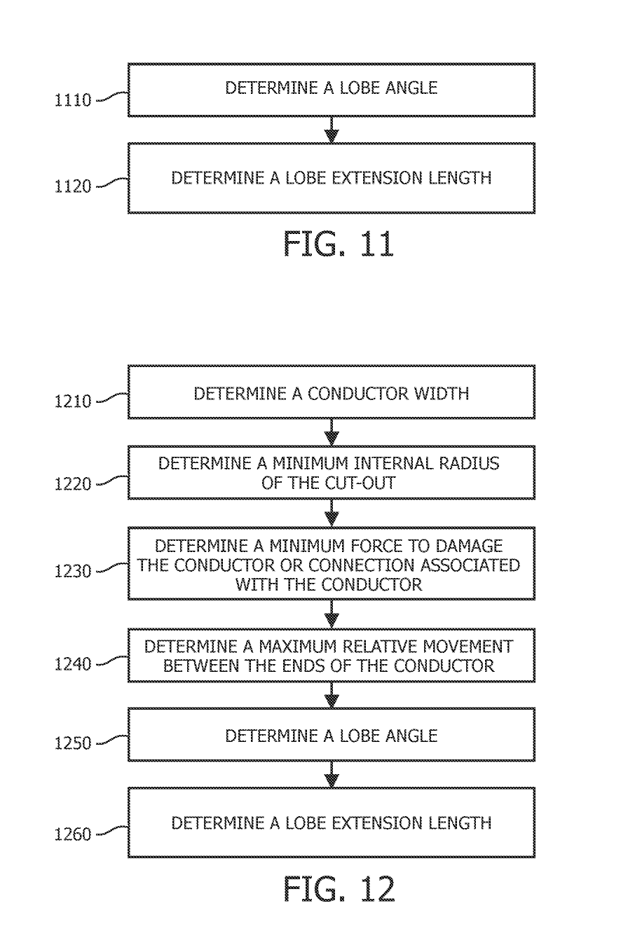

FIG. 11 is a flowchart of an exemplary method of designing an exemplary flexible electronic pathway;

FIG. 12 is a flowchart of another exemplary method of designing an exemplary flexible electronic pathway;

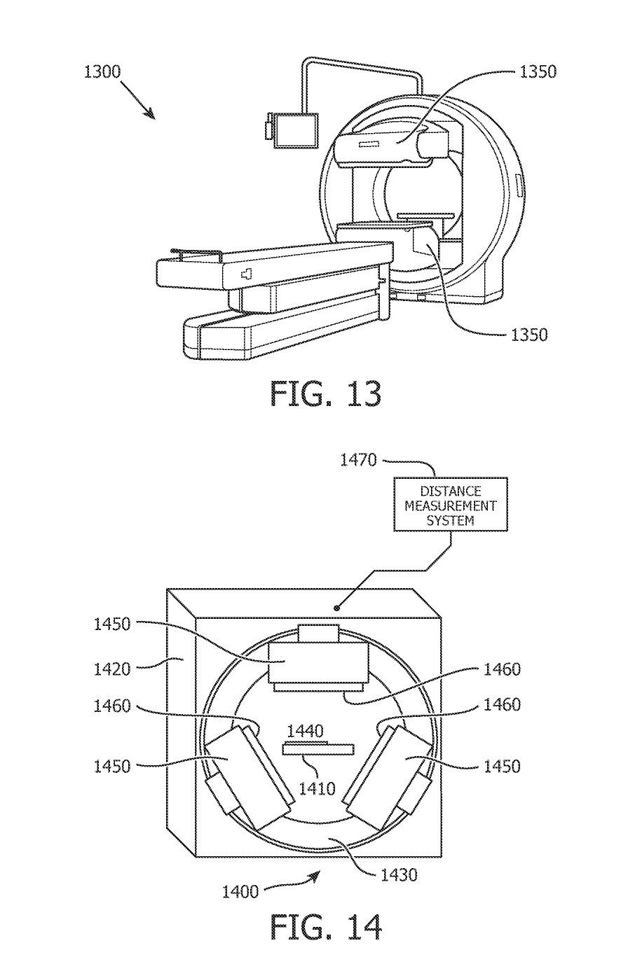

FIG. 13 shows an exemplary imaging apparatus;

FIG. 14 illustrates another exemplary imaging apparatus with a partial block diagram;

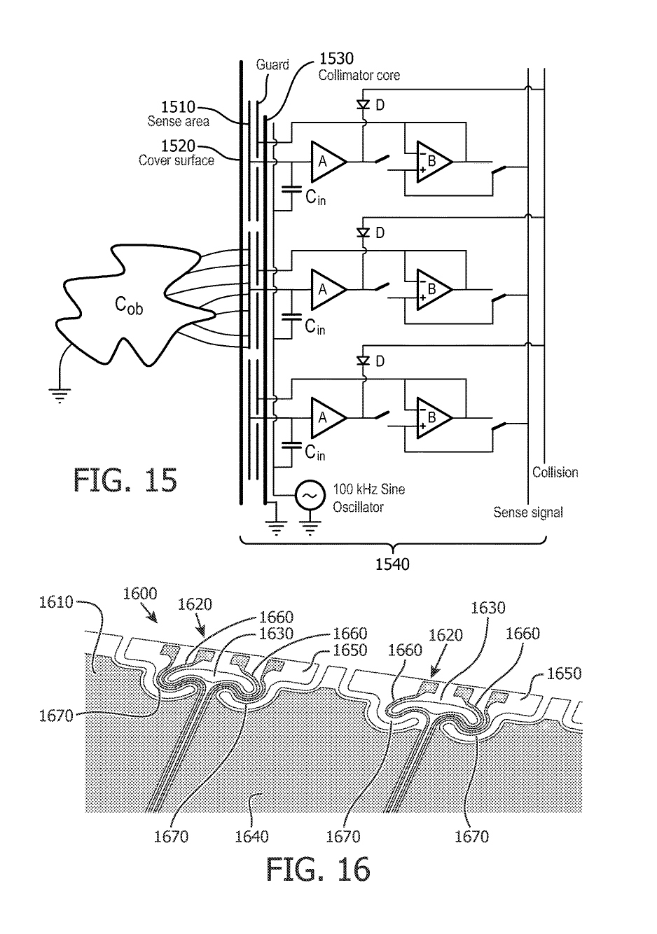

FIG. 15 illustrates exemplary electronics of an exemplary subject locating system of an imaging apparatus; and

FIG. 16 illustrates an exemplary flexible electronic pathway as part of an exemplary sensor sheet.

In one embodiment, an exemplary flexible electronic pathway 100, which may be, for example, a FFC, is shown in FIG. 1. In this embodiment, the pathway 100 includes a flat conductor 110 to connect a first device 112 to a second device 114. Devices 112, 114 may be any electronic device or pathway, including, for example, a PCB, sensor, cable, connector, conductor, etc. The flat conductor 110 is shown terminating a first end 116 of the flat conductor 110 to the first device 112 at termination point 118. The flat conductor 110 is also shown terminating a second end 120 of the flat conductor 110 to the second device 114 at termination point 122. The termination points 118, 122 may be any type of termination, including, for example, connectors or connections directly to the devices 112, 114 using, for example, adhesive, ACF tape, and other mechanical and/or electronic terminations and connections.

The exemplary flat conductor 110 includes an insulative substrate and at least one electrical conductor, including, for example, a wire of a flexible cable, a trace of a flexible circuit (see, e.g., traces 1660 shown in FIG. 16), combinations thereof, etc. In one embodiment of a flat conductor 110, a flexible flat cable may include a plurality of wires, each surrounded by insulation. In another embodiment of a flat conductor 110, a flexible circuit may include a plurality of traces on one or both sides of an insulating sheet. In yet another embodiment of a flat conductor 110, the flat conductor 110 may be multi-layered. FIG. 1 shows an exemplary flexible electronic pathway 100 with a flat conductor 110 with a relatively short conductor length between devices. In other embodiments, the length of the flat conductor 110 may be relatively long before terminating.

The exemplary flat conductor 110 also includes a cut-out 130 within the flat conductor 110. The cut-out 130 is an opening within the flat conductor 110 that provides strain and/or stress relief to the electrical pathway 100 and balances the forces applied to the termination sites 118, 122 during relative movement between the ends 116, 120 of the flat conductor 110. As shown in this embodiment, the cut-out 130 may be a relatively narrow opening centered laterally in the flat conductor 110 and extending outward on both sides into two symmetrical side lobes 140 of the flat conductor 110. At the cut-out 130, the flat conductor 110 splits into two conductor portions 142, 144 that route the electrical conductors around the cut-out 130. In one embodiment, the two conductor portions 142, 144 preferably stay close to the cut-out 130 contour to minimize electrical conductor length, overall size, etc.

Several cut-out 130 shapes are effective at providing strain relief and balancing stress. For reference, the exemplary electronic pathway 100 is shown with the flat conductor 110 in an X-Y plane, where the electrical conductors generally route in the Y direction between ends 116, 120. In one embodiment, for example, the cut-out 130 and lobes 140 may extend straight out in the +X and −X directions to accommodate lateral movement (relative movement between the ends 116, 120 in the X direction). However, such shapes may not accommodate much axial movement (extension and contraction relative movement between the ends 116, 120 in the Y direction) unless the cut-out 130 and lobes 140 are relatively long. Some amount of vertical movement (relative movement between the ends 116, 120 in the Z direction) is also accommodated. In another embodiment, the cut-out 130 and the lobes 140 include a curved portion, as shown in FIG. 1 as portions 142, 144, to better accommodate lateral, axial, and vertical relative movements. The features and components described above in relation to the embodiment of FIG. 1 may also apply to other embodiments described below.

In a similar embodiment, an exemplary flexible electronic pathway 200 is shown in FIG. 2. In this embodiment, the pathway 200 includes a flat conductor 210 to connect the first device 112 to the second device 114. The flat conductor 210 is shown terminating a first end 216 of the flat conductor 210 to the first device 112 at termination point 118. The flat conductor 210 is also shown terminating a second end 220 of the flat conductor 210 to the second device 114 at termination point 122. The exemplary flat conductor 210 also includes a cut-out 230 within the flat conductor 210. Similar to cut-out 130, the cut-out 230 provides strain and/or stress relief to the electrical pathway 200 and balances the forces applied to the termination sites 118, 122 during relative movement between the ends 216, 220 of the flat conductor 210. The cut-out 230 extends outward on both sides into two symmetrical side lobes 240 of the flat conductor 210. At the cut-out 230, the flat conductor 210 splits into two conductor portions 242, 244.

As shown in FIG. 2, the cut-out 230 and the lobes 240 include longer extensions than cut-out 130 and lobes 140 of FIG. 1 to provide additional strain relief and balancing. In particular, the longer cut-out 230 and lobes 240 allow pathway 200 to accommodate more translational relative movement between the ends 216, 220 of the flat conductor 210 in lateral (X), axial (Y), and vertical (Z) directions, and combinations thereof. However, in addition to accommodating translational relative movement, pathways 100, 200, and others mentioned below are also able to accommodate rotational relative movement about the three axes (X, Y, and Z) and combinations thereof.

In another embodiment, an exemplary flexible electronic pathway 300 is shown in FIG. 3. In this embodiment, the pathway 300 includes a flat conductor 310 to connect the first device 112 to the second device 114. The flat conductor 310 is shown terminating a first end 316 of the flat conductor 310 to the first device 112 at termination point 118. The flat conductor 310 is also shown terminating a second end 320 of the flat conductor 310 to the second device 114 at termination point 122. The exemplary flat conductor 310 also includes a cut-out 330 within the flat conductor 310. The cut-out 330 provides strain and/or stress relief to the electrical pathway 300 and balances the forces applied to the termination sites 118, 122 during relative movement between the ends 316, 320 of the flat conductor 310. The cut-out 330 extends outward on both sides into two symmetrical side lobes 340 of the flat conductor 310. At the cut-out 330, the flat conductor 310 splits into two conductor portions 342, 344. As shown in FIG. 3, the cut-out 330 and each of the lobes 340 may include bifurcated portions and multiple nodes.

In another embodiment, an exemplary flexible electronic pathway 400 is shown in FIG. 4. In this embodiment, the pathway 400 includes a flat conductor 410 to connect the first device 112 to the second device 114. The flat conductor 410 is shown terminating a first end 416 of the flat conductor 410 to the first device 112 at termination point 118. The flat conductor 410 is also shown terminating a second end 420 of the flat conductor 410 to the second device 114 at termination point 122. The exemplary flat conductor 410 also includes a cut-out 430 within the flat conductor 410. The cut-out 430 provides strain and/or stress relief to the electrical pathway 400 and balances the forces applied to the termination sites 118, 122 during relative movement between the ends 416, 420 of the flat conductor 410. The cut-out 430 extends outward on both sides into two symmetrical side lobes 440 of the flat conductor 410. At the cut-out 430, the flat conductor 410 splits into two conductor portions 442, 444. As shown in FIG. 4, the cut-out 430 and each of the lobes 440 may include bifurcated portions.

In another embodiment, an exemplary flexible electronic pathway 500 is shown in FIG. 5. In this embodiment, the pathway 500 includes a flat conductor 510 to connect the first device 112 to a second device 514. The flat conductor 510 is shown terminating a first end 516 of the flat conductor 510 to the first device 112 at termination point 118. The flat conductor 510 is also shown terminating a second end 520 of the flat conductor 510 to the second device 514 at termination point 522. As can be seen in FIG. 5, the second end 520 that terminates to the second device 514 at termination point 522 is not in-line with the first end 516 that terminates to the first device 112 at termination point 118. The ends 516, 520 are shown offset or rotated approximately 90 degrees in the X-Y plane. The exemplary flat conductor 510 also includes a cut-out 530 within the flat conductor 510. The cut-out 530 provides strain and/or stress relief to the electrical pathway 500 and balances the forces applied to the termination sites 118, 522 during relative movement between the ends 516, 520 of the flat conductor 510. The cut-out 530 extends outward on both sides into two non-symmetrical side lobes 540 of the flat conductor 510. At the cut-out 530, the flat conductor 510 splits into two conductor portions 542, 544. As shown in FIG. 5, the cut-out 530 and each of the lobes 540 may include several non-symmetrical features. As can be appreciated, a flexible electronic pathway can be configured for virtually any application, including, for example, applications where one or more ends are offset, rotated, staggered, mis-aligned, etc. from each other.

As can be appreciated by the embodiments shown in FIGS. 1-5, many different configurations not shown, including, for example, various shapes and sizes of flexible electronic pathways with cut-outs can also be used. Variations in design do not depart from the scope of the invention, including, for example, changes to the width of the cut-out area, offsetting the cut-out shape and/or lobes laterally, adding multiple nodes in series, flipping the pattern around, creating non-symmetric cut-out and/or lobe shapes, changing the cut-out contour path from curved with an extension to a combination of other shapes, off-setting one or more ends, etc. The various design patterns may also be embedded into a surrounding sheet. While not always optimal in terms of space, such design variations may be required to avoid obstacles and/or to position parts of the pathway where space is available in the design and/or application for routing, bending, twisting, etc.

All of these designs, including pathways 100, 200, 300, 400, 500 shown in FIGS. 1-5, can accommodate various degrees of strain and stress relief and balance the forces applied to the termination sites 118, 122, 522, including during translational relative movement in lateral (X), axial (Y), and vertical (Z) directions, rotational relative movement about the lateral (X), axial (Y), and vertical (Z) axes, and combinations thereof. The pathways are able to gradually absorb the strain and/or stress applied to the pathway by distributing the force throughout the pathway portions. In particular, when a force is applied to a pathway, such as, for example, pathways 100, 200, 300, 400, 500, portions of the pathway, such as, for example, the flat conductors 110, 210, 310, 410, 510 including the lobes 140, 240, 340, 440, 540 may change shape into a tense state, for example, by distorting, twisting, lifting, bending, etc. from their nominal relaxed state. This shape changing response to a force is elastic and can generate a spring-like reaction force responsible for the effective compliance. Because the design in the relaxed state represents the center of the compliant space, displacements in positive and negative directions are accommodated.

FIGS. 6-8 show several exemplary embodiments of flexible electronic pathways in various states accommodating relative movements between conductor ends.

For example, FIGS. 6A-6C show an exemplary flexible electronic pathway 600. In this embodiment, the pathway 600 includes a flat conductor 610 to connect a first device 612 to a second device 614. The flat conductor 610 is shown terminating a first end 616 of the flat conductor 610 to the first device 612 at termination point 618. The flat conductor 610 is also shown terminating a second end 620 of the flat conductor 610 to the second device 614 at termination point 622. The exemplary flat conductor 610 also includes a cut-out 630 within the flat conductor 610. The cut-out 630 provides strain and/or stress relief to the electrical pathway 600 during relative movement between the ends 616, 620 of the flat conductor 610. The cut-out 630 extends outward on both sides into two symmetrical side lobes 640 of the flat conductor 610. At the cut-out 630, the flat conductor 610 splits into two conductor portions 642, 644. In this embodiment, the cut-out 630 and each of the side lobes 640 include a curved portion.

FIG. 6A shows the exemplary pathway 600 in a relaxed state that represents the center of the compliant space or range. In this state there is no relative movement between the ends 616, 620 of the flat conductor 610. The flat conductor 610 is generally flat within the X-Y plane. FIG. 6B shows the exemplary pathway 600′ in a tense state in response to translational axial relative movement between the ends 616, 620. In particular, device 614 is shown after moving in the +Y direction towards device 612 with a force causing relative movement between the ends 616, 620. It should be noted that if device 612 moves in the −Y direction towards device 614 with the same force, the same relative movement is created. In response to this relative movement, the shape of the pathway 600′ is different than the pathway 600 of FIG. 6A. In particular, portions of the flat conductor 610′ have changed shape and no longer remain flat within the X-Y plane. For example, portions of the flat conductor 610′ without the cut-out 630 have raised up in the +Z direction and side lobes 640′ are raised in the +Z direction and are twisted and bent along the split conductor portions 642′, 644′ surrounding the cut-out 630. FIG. 6C shows the exemplary pathway 600″ in another tense state in response to even more translational axial relative movement between the ends 616, 620. In particular, device 614 is shown after moving more in the +Y direction towards device 612 with a force causing more relative movement between the ends 616, 620. In response to this relative movement, the shape of the pathway 600″ is different than the pathway 600′ of FIG. 6B. In particular, portions of the flat conductor 610″ have changed shape even more and are further from being flat within the X-Y plane. For example, portions of the flat conductor 610″ without the cut-out 630 have raised up more in the +Z direction and side lobes 640″ are raised more in the +Z direction and are twisted and bent more along the split conductor portions 642″, 644″ surrounding the cut-out 630. In this state, the edges of the cut-out 630 are almost overlapping in the Y direction. However, both pathways 600′, 600″ are able to absorb the strain and stress created by the relative movement between the ends 616, 620 without causing any damage to the conductor 610 (shown as 610′, 610″ in the tense states), termination points 618, 622, or devices 612, 614.

In another embodiment, FIGS. 7A-7C show an exemplary flexible electronic pathway 700. In this embodiment, the pathway 700 includes a flat conductor 710 to connect a first device 712 to a second device 714. The flat conductor 710 is shown terminating a first end 716 of the flat conductor 710 to the first device 712 at termination point 718. The flat conductor 710 is also shown terminating a second end 720 of the flat conductor 710 to the second device 714 at termination point 722. The exemplary flat conductor 710 also includes a cut-out 730 within the flat conductor 710. The cut-out 730 provides strain and/or stress relief to the electrical pathway 700 during relative movement between the ends 716, 720 of the flat conductor 710. The cut-out 730 extends outward on both sides into two symmetrical side lobes 740 of the flat conductor 710. At the cut-out 730, the flat conductor 710 splits into two conductor portions 742, 744. In this embodiment, the cut-out 730 and each of the side lobes 740 include a curved portion.

FIG. 7A shows the exemplary pathway 700 in a tense state in response to translational lateral relative movement between the ends 716, 720. In particular, device 714 is shown after moving in the −X direction with a force causing relative movement between the ends 716, 720. In response to this relative movement, the shape of the pathway 700 is different than the shape of the pathway 700 when in a relaxed state, for example, similar to pathway 600 as shown in FIG. 6A. In particular, portions of the flat conductor 710 have changed shape and no longer remain flat within the X-Y plane. For example, side lobes 740 are bent down in the −Z direction. FIG. 7B shows the exemplary pathway 700′ in another tense state in response to translational lateral relative movement between the ends 716, 720. In particular, device 714 is shown after moving more in the −X direction with a force causing more relative movement between the ends 716, 720. In response to this relative movement, the shape of the pathway 700′ is different than the pathway 700 of FIG. 7A. In particular, portions of the flat conductor 710′ have changed shape even more and are further from being flat within the X-Y plane. For example, side lobes 740′ are bent more in the −Z direction and are twisted and bent along the split conductor portions 742′, 744′ surrounding the cut-out 730. FIG. 7C shows the exemplary pathway 700″ in another tense state in response to translational lateral relative movement and rotational relative movement between the ends 716, 720. In particular, device 714 is shown after moving in the +X direction and rotating about the Y-axis with a force causing relative movement between the ends 716, 720. In response to this relative movement, the shape of the pathway 700″ is different than the pathways 700, 700′ of FIGS. 7A and 7B. In particular, portions of the flat conductor 710″ have changed shape even more and are further from being flat within the X-Y plane. For example, side lobes 740″ are bent more in the −Z direction and are more twisted and bent along the split conductor portions 742″, 744″ surrounding the cut-out 730. However, all of the pathways 700, 700′, 700″ are able to absorb the strain and stress created by the relative movement between the ends 716, 720 without causing any damage to the conductor 710 (also shown as 710′, 710″), termination points 718, 722, or devices 712, 714.

In yet another embodiment, FIGS. 8A-8B show an exemplary flexible electronic pathway 800. In this embodiment, the pathway 800 includes a flat conductor 810 to connect a first device 812 to a second device 814. The flat conductor 810 is shown terminating a first end 816 of the flat conductor 810 to the first device 812 at termination point 818. The flat conductor 810 is also shown terminating a second end 830 of the flat conductor 810 to the second device 814 at termination point 822. The exemplary flat conductor 810 also includes a cut-out 830 within the flat conductor 810. The cut-out 830 provides strain and/or stress relief to the electrical pathway 800 during relative movement between the ends 816, 820 of the flat conductor 810. The cut-out 830 extends outward on both sides into two symmetrical side lobes 840 of the flat conductor 810. At the cut-out 830, the flat conductor 810 splits into two conductor portions 842, 844. In this embodiment, the cut-out 830 and each of the side lobes 840 include a curved portion.

FIG. 8A shows the exemplary pathway 800 in a tense state in response to translational axial relative movement between the ends 816, 820. In particular, device 814 is shown after moving in the −Y direction with a force causing relative movement between the ends 816, 820. In response to this relative movement, the shape of the pathway 800 is different than the shape of the pathway 800 when in a relaxed state, for example, similar to pathway 600 as shown in FIG. 6A. In particular, portions of the flat conductor 810 have changed shape and no longer remain flat within the X-Y plane. For example, side lobes 840 are bent down in the −Z direction. FIG. 8B shows the exemplary pathway 800′ in another tense state in response to translational axial relative movement and rotational relative movement between the ends 816, 820. In particular, device 814 is shown after moving in the −Y direction and rotating about the Z-axis with a force causing more relative movement between the ends 816, 820. In response to this relative movement, the shape of the pathway 800′ is different than the pathway 800 of FIG. 8A. In particular, portions of the flat conductor 810′ have changed shape even more and are further from being flat within the X-Y plane. For example, portions of the flat conductor 810′ without the cut-out 830 have bent in the Z direction and side lobes 840′ are bent more in the −Z direction and are twisted and bent along the split conductor portions 842′, 844′ surrounding the cut-out 830. However, both of the pathways 800, 800′ are able to absorb the strain and stress created by the relative movement between the ends 816, 820 without causing any damage to the conductor 810 (also shown as 810′), termination points 818, 822, or devices 812, 814.

In some embodiments, one element of the design of a pathway may be to establish a minimum internal radius of the cut-out area and external shapes, such as, for example, side lobes. Establishing this radius reduces stress concentrations, for example, by avoiding sharp corners and reducing the risk of tears in the pathway due to fatigue, high stress events, etc. This radius may be chosen, for example, based on the minimum practical radius due to the manufacturing method of the pathway. Larger radii may spread out the forces that incur twisting of the pathway. In one embodiment, the cut-outs can maintain the diameter of these corners (based on the established radius) as their gap width to avoid interference between adjacent materials during movement, especially compression, for example. In other embodiments the gap can be reduced as desired and the conductors routed accordingly.

In other embodiments, the design of a pathway may be tuned to control the amount of mechanical resistance. When pulling on one end of the pathway, while the other end of the pathway remains fixed, a gradually rising opposing force can develop while the strain relief feature of the pathway elastically twists and distorts its physical shape in response to the force. The resistance is the reaction force divided by the distance, similar to a spring constant. In certain embodiments, the resistance may be tuned, for example, by adjusting two design parameters: 1) a strain relief or lobe angle alpha; and 2) a side-lobe or simply lobe extension length d_ext. In one embodiment, an angle alpha=0° (straight lateral extension) and d_ext=0, which has minimal compliance, where compliance is the ability of the pathway to allow the relative movement between the ends of the pathway without damage by absorbing or accommodating the applied force. In another embodiment, an angle of alpha=90° and d_ext>0, which has a maximum axial compliance. In various other embodiments, intermediate alpha angles provide different relative amounts of lateral and axial compliance. The desired direction and degree of compliance may be specific to each application.

One embodiment of designing a pathway includes determining the minimum amount of force necessary to damage or compromise an electrical connection associated with an end of the pathway and establishing a safety factor below this as the maximum allowable force. In other embodiments, torque due to applied moments may also be considered as a factor. The maximum expected displacement and rotation based on mechanical movement, thermal expansion, service requirements, etc. may also be determined. From these two determinations, various alpha angles and side-lobe extensions of the pathway design may be prototyped using comparable materials cut into representative shapes and tested until a pathway design is identified that reacts with the maximum allowed force when shifted the maximum displacement. This method is important for minimizing the size of the pathway features and for reliability.

In one embodiment, other variables that may be included in the design of a pathway are the pathway or main cable width w1, the split portion or cable width w2 (the width of the cable after it splits in two around the cut-out), and d_cut (the width of the cut-out and diameter of the fillets on all internal and external edges). To minimize the size of the strain relief area of the pathway, d_cut may be chosen based on the minimum radius that can be manufactured using standard production methods. In one embodiment, all other features of the pathway design are dependent only on these variables. The origin of the design determines the location of the strain relief cut-out feature the pathway.

In one embodiment, an exemplary flexible electronic pathway 900 is shown in FIG. 9 as a flat cable. In this embodiment, the pathway 900 includes a flat conductor 910 to connect a first device 912 to a second device 914. The flat conductor 910 is shown terminating a first end 916 of the flat conductor 910 to the first device 912 at termination point 918. The flat conductor 910 is also shown terminating a second end 920 of the flat conductor 910 to the second device 914 at termination point 922. The exemplary flat conductor 910 also includes a cut-out 930 within the flat conductor 910. The cut-out 930 provides strain and/or stress relief to the electrical pathway 900 during relative movement between the ends 916, 920 of the flat conductor 910. The cut-out 930 extends outward on both sides into two symmetrical side lobes 940 of the flat conductor 910. At the cut-out 930, the flat conductor 910 splits into two conductor portions 942, 944.

In this embodiment, the design of the pathway 900 demonstrates how a single point A can serve as the origin for all of the pathway 900 features. As shown in FIG. 9, the cable width w1=24, the split-width of the cable w2=12, the minimum inside diameter d_cut=4 (shown as a radius of 2), the lobe or strain relief angle alpha=60°, and the lobe extension length d_ext=7. All dimensions shown in FIG. 9 are combinations of or derived from these parameters. For example, the R30 includes (d_int/2+w2+d_int+w2)=(4/2+12+4+12). All lines and arcs are defined to be tangent where they join. In this embodiment, the left side is defined to be symmetrical to the right side.

As discussed above, variations can include changes to the width of the cut-out, offsetting the cut-out shape laterally, adding multiple nodes, flipping the pattern around, creating a non-symmetric shapes, and changing the cut out contour path from curved with extension to a combination of other shapes.

For example, another exemplary flexible electronic pathway 1000 is shown in FIG. 10 as another flat cable. In this embodiment, the pathway 1000 includes a flat conductor 1010 to connect a first device 1012 to a second device 1014. The flat conductor 1010 is shown terminating a first end 1016 of the flat conductor 1010 to the first device 1012 at termination point 1018. The flat conductor 1010 is also shown terminating a second end 1020 of the flat conductor 1010 to the second device 1014 at termination point 1022. The exemplary flat conductor 1010 also includes a cut-out 1030 within the flat conductor 1010. The cut-out 1030 provides strain and/or stress relief to the electrical pathway 1000 during relative movement between the ends 1016, 1020 of the flat conductor 1010. The cut-out 1030 extends outward on both sides into two symmetrical side lobes 1040 with multiple nodes. At the cut-out 1030, the flat conductor 1010 splits into two conductor portions 1042, 1044.

As shown in FIG. 10, the cable width w1=24, the split-width of the cable w2=12, the minimum inside diameter d_cut=4 (shown as a radius of 2), the lobe or strain relief angle alpha=57°, and the lobe extension length d_ext=3. All dimensions shown in FIG. 10 are combinations of or derived from these parameters. All lines and arcs are defined to be tangent where they join. In this embodiment, the left side is defined to be symmetrical to the right side.

The design shown in the embodiment of FIG. 10 is valid for angles of alpha greater than 0° and less than 90°. However, the definition of the two curves along the horizontal centerline 1050 of the pattern changes at about angles of 57° in this embodiment. For alpha angles between 0° and 57°, the arc dimensioned with the R14 radius shown just above the horizontal centerline 1050 on the right side has a fixed radius of w2+d_cut/2 (=14 in this example), has ends constrained to be tangent to the 180 degree semi-circular prongs of the design. For angles between 57° and 90°, the center of this R14 arc and the smaller concentric arc spanning the centerline are tied to the intersection of the d_ext offset construction line (dimensioned as 3) with the horizontal pattern center line, moving outward as angle alpha approaches 0, so R14 grows larger than 14. This is to maintain consistency with design rules previously established and prevent crossovers of the patterns. Other designs may have similar characteristic dependencies at different parametric values.

In general, by adding an in-line strain relief cut-out feature in the pathway, for example, close to the termination site, a person pulling on the pathway will sense a gradually increasing amount of resistance, signaling them to reduce their effort and limiting potential disconnection or damage to the termination. The contours in the pathway act like a spring and limit the rate of change of the applied stress, reducing damaging sharp, hard forces, mechanical shock, etc. applied on the connection.

FIGS. 11-12 describe exemplary methods associated with designing flexible electronic pathways, including, for example, those mentioned above. Further embodiments of similar methods may include other additional steps, or omit one or more of the steps in the illustrated methods. Also, the order in which the process flows herein have been described may be rearranged while still accomplishing the same results. Thus the process flows described herein may be added to, rearranged, consolidated, and/or re-organized in their implementation as warranted or desired.

FIG. 11 is a flowchart of an exemplary method of designing a flexible electronic pathway based on two key features. At step 1110, a lobe angle is determined. At step 1120, a lobe extension length is determined. As described above, a pathway design can be developed from these parameters.

FIG. 12 is a flowchart of another exemplary method of designing a flexible electronic pathway based on the same key features, along with other design considerations. At step 1210, a conductor width is determined. At step 1220, a minimum internal radius of the cut-out is determined. At step 1230, a minimum force to damage the conductor or connection associated with the conductor is determined. At step 1240, a maximum relative movement between the ends of the conductor is determined. At step 1250, a lobe angle may be determined based on one or more of the above parameters. At step 1260, a lobe extension length may be determined based on one or more of the above parameters. In other embodiments, one or more of the above steps may be re-ordered, repeated, skipped, and/or augmented.

When positioned near a termination, the symmetric design of the inline strain relief cut-out effectively isolates a device like an island connected by flexible bridges to the main device, cable, PCB, etc. This helps distribute stress over the entire termination area rather than concentrating the stress on one side or another as would be experienced by a termination without strain relief subjected to off-axis forces. The cut-out design also represents an improvement over one-sided bends that preferentially transfer stress to one side.

Various embodiments, including those described above, can provide significant amounts of compliance in lateral, axial, and vertical directions, in rotation about each direction, and combinations thereof. This is substantially different than designs and methods that include slack or bends in cables, which provide compliance in only one or two directions. In addition, the cut-out design does not require external means of fixation of the terminations, thereby allowing servicing or upgrading of components without having to re-apply adhesives or remove cables from fixation devices, for example.

The described pathways and methods of determining the required amount of compliance help minimize the overall size required for the pathway and cut-out design. The size may be tightly constrained by the minimum radius of the cutouts, the width of the full and split sections of flexible conductor, and/or the radius and extension distance chosen to meet the mechanical resistance requirements of any particular application.

Any electronics assembly employing electrical pathways, including, for example, FFCs such as flat flexible cables and/or flex circuits, is a potential application for a pathway with an in-line strain relief cut-out. Assemblies that may include such pathways include, for example, notebook and tablet computers, mobile phones, LCD televisions and displays, etc. Flexible electronic pathways are commonly incorporated into many kinds of portable instrumentation and equipment including medical equipment and high-end consumer electronics.

For example, one embodiment includes a sensor array for the Auto Body Contouring function of the Philips BrightView SPECT imaging systems (available from Philips Medical Systems). An exemplary imaging system 1300 is shown in FIG. 13 with detector heads 1350, which contain the sensor arrays. The medical diagnostic imaging system and apparatus 1300 can detect and record the spatial, temporal, and/or other characteristics of emitted photons.

More specifically, in one exemplary embodiment, with reference to FIG. 14, the diagnostic nuclear imaging apparatus or scanner 1400 is a SPECT imaging system. The illustrated exemplary SPECT imaging system 1400 is a Philips BrightView SPECT system. The SPECT imaging system 1400 includes a subject support 1410, such as a table or couch, which supports and positions a subject being examined and/or imaged, such as a phantom or patient. A stationary gantry 1420 may also hold a rotating gantry 1430 mounted thereto. The gantry 1420 defines a subject-receiving aperture 1440. One or more detector heads 1450 are mounted to the gantry 1420 (or rotating gantry 1430). The rotating gantry 1430 and the detector heads 1450 may be adapted to rotate about the subject-receiving aperture 1440 (and the subject when located therein).

Each of the detector heads 1450 has a radiation-receiving face adapted to face the subject-receiving aperture 1440. The detector heads 1450 include collimators 1460 mounted on the radiation receiving faces of the detector heads 1450. The collimators 1460 may include position sensor arrays on the faces and sides near the collimator surface. These sensor arrays are part of a subject location system, for locating the detector head 1450 near the subject, that includes a distance measurement system 1470.

With further reference to FIG. 15, which shows exemplary electronics associated with the subject location system, a sensor array 1510 may be positioned between a collimator cover 1520 and a collimator core 1530. The Philips Auto Body Contouring system measures a capacitance of the object (C_{Ob}) to be scanned near the collimator 1450 to determine the distance from the collimator cover 1520 to the object using the distance measurement system 1470 and associated sensing electronics 1540. The sensing electronics 1540 may be located in the collimator 1450 or any associated device.

In this embodiment, and with further reference to FIG. 16, which shows an exemplary flexible electronic pathway, shown as flat conductor/sensor sheet 1600, associated with the subject location system, connections for the sensors 1610 may be routed to the closest edges of the sensor sheet 1600 where they can attach to devices, such as, for example, PCBs (not shown). As shown, a flexible electronic pathway 1620 with cut-out 1630, in accordance with any of the designs mentioned above, allows relative movement between ends 1640, 1650 of the sheet 1600, which may be due to differential thermal expansion, flexing, handling, etc. of the cover, without changing the overall size of the sensor sheet 1600. The flexible electronic pathway 1620 routes electrical conductors 1660, in the form of electrical traces, around cut-out 1630 through lobes 1670.

While the present invention has been illustrated by the description of embodiments thereof, and while the embodiments have been described in some detail, it is not the intention of the applicant to restrict or in any way limit the scope of the appended claims to such detail. Additional advantages and modifications will readily appear to those skilled in the art. The invention may take form in various compositions, components and arrangements, combinations and sub-combinations of the elements of the disclosed embodiments. Therefore, the invention in its broader aspects is not limited to the specific details, representative apparatus and methods, and illustrative examples shown and described. Accordingly, departures may be made from such details without departing from the spirit or scope of the applicant's general inventive concept.

1. A flexible electronic pathway, comprising:
a flat conductor comprising:
an electrical conductor; and
an insulative substrate; and
a cut-out within a portion of the flat conductor to provide strain relief, wherein the cut-out is surrounded by side lobes extending laterally in opposite directions from the axial path of the flat conductor;
wherein the flexible electronic pathway allows a relative movement between a first end and a second end of the flat conductor.

2. The flexible electronic pathway of claim 1, wherein the flat conductor comprises a flexible circuit.

3. The flexible electronic pathway of claim 1, wherein the flat conductor comprises a flexible cable.

4. The flexible electronic pathway of claim 1, wherein the flat conductor comprises a connector.

5. The flexible electronic pathway of claim 4, wherein the connector is connected to a printed circuit board at the first end or the second end.

6. The flexible electronic pathway of claim 1, wherein the flexible electronic pathway gradually increases a resistance to a force causing the relative movement as the relative movement increases.

7. The flexible electronic pathway of claim 1, wherein the relative movement comprises a linear movement in an axial plane.

8. The flexible electronic pathway of claim 1, wherein the relative movement comprises at least two of a linear movement in a lateral plane, an axial plane, and a vertical plane.

9. The flexible electronic pathway of claim 1, wherein the relative movement comprises a rotational movement about a lateral axis, an axial axis, or a vertical axis.

10. The flexible electronic pathway of claim 1, wherein the relative movement comprises at least two of a rotational movement about a lateral axis, an axial axis, and a vertical axis.

11. The flexible electronic pathway of claim 1, wherein the relative movement comprises a linear movement in a lateral plane, an axial plane, or a vertical plane and a rotational movement about a lateral axis, an axial axis, or a vertical axis.

12. The flexible electronic pathway of claim 1, wherein the cut-out is centered laterally within the flat conductor.

13. The flexible electronic pathway of claim 1, wherein the side lobes are symmetrical.

14. The flexible electronic pathway of claim 13, wherein the lateral side lobes are curved.

15. A medical diagnostic imaging apparatus, comprising:
a gantry with an aperture for receiving a subject;
at least one detector head mounted to the gantry for receiving radiation;
a subject location system for locating the at least one detector near the subject, wherein the subject location system comprises:
a sensor array for sensing a condition associated with a location of the at least one detector relative to the subject;
a distance measurement system for determining a distance from the at least one detector to the subject; and
a flexible electronic pathway for providing electronic communications between the sensor array and the distance measuring system, wherein the flexible electronic pathway comprises:
a flat conductor comprising:
an electrical conductor; and
an insulative substrate; and
a cut-out within a portion of the flat conductor to provide strain relief, wherein the cut-out is surrounded by side lobes extending laterally in opposite directions from the axial path of the flat conductor;
wherein the flexible electronic pathway allows a relative movement between a first end and a second end of the flat conductor.

16. A method of designing a flexible electronic pathway that comprises a flat conductor and a cut-out within the flat conductor to provide strain relief, such that the flexible electronic pathway allows relative movement between a first end and a second end of a flat conductor, comprising:
determining a lobe angle, wherein the lobe angle defines a curved portion of the flat conductor surrounding the cut-out; and
determining a lobe extension length, wherein the lobe extension length defines a straight portion of the flat conductor surrounding the cut-out.

17. The method of designing a flexible electronic pathway according to claim 16, further comprising:
determining a width of the flat conductor; and
determining a minimum internal radius of the cut-out.

18. The method of designing a flexible electronic pathway according to claim 16, further comprising determining a minimum force to damage an electrical connection associated with the first end or the second end, wherein determining the lobe angle and determining the lobe extension length is based on the minimum force.

19. The method of designing a flexible electronic pathway according to claim 16, further comprising determining a maximum relative movement between the first end and the second end, wherein determining the lobe angle and determining the lobe extension length is based on the maximum relative movement.

20. The method of designing a flexible electronic pathway according to claim 19, wherein the maximum relative movement comprises at least one of a linear movement in a lateral plane, an axial plane, or a vertical plane and a rotational movement about a lateral axis, an axial axis, or a vertical axis.
